(19) United States
(12) Patent Application Publication
(10) Pub. No.: US 2006/0079848 A1
(43) Pub. Date: Apr. 13, 2006

(54) Non-skin penetrating reconstituting syringe

(75) Inventors: Brian J. Pelkey, Rockaway, NJ (US); Julia E. Griggs, San Jose, CA (US)

Correspondence Address: DAVID W. HIGHET, VP AND CHIEF IP COUNSEL;BECTON, DICKINSON AND COMPANY, 1 BECTON DRIVE, MC 110, FRANKLIN LAKES, NJ, 07417-1880, US

(73) Assignee: Becton, Dickinson and Company, Franklin Lakes, NJ (US)

(21) Appl. No.: 10/879,932

(22) Filed: Jun. 29, 2004

(51) Int. Cl. A61M 5/31; A61M 5/32

(52) U.S. Cl. 604/240; 604/272

(57) ABSTRACT

A reconstitution syringe assembly comprises an elongate cannula made of thermoplastic material having an outside diameter of at least 2 mm, a distal tip capable of piercing an elastomeric vial stopper and a proximal end having discontinuities on its exterior surface. A barrel made of thermoplastic material includes a tip which is molded in intimate contact around the proximal end of the cannula engaging the discontinuities to provide a permanently attached cannula. The cannula extends at least 23 mm from the distal wall of the barrel. An integrally formed one-piece thermoplastic plunger and stopper is provided.

### BACKGROUND OF THE INVENTION

The present invention relates to syringe assemblies and more particularly concerns disposable syringe and cannula assemblies used to reconstitute medication for delivery.

Throughout the world, multiple use of hypodermic syringe products, which are intended for single-use only, is instrumental in drug abuse and in the transfer of contagious diseases. Intravenous drug users who routinely share and reuse needles are a high-risk group with respect to the AIDS virus and hepatitis. Also, the effects of multiple use are a concern in some countries where repeated use of syringe products during mass immunization programs may be responsible for the spread of many diseases. Re-use of single-use hypodermic products is also instrumental in the spread of drug abuse even in the absence of infection or disease.

In mass immunization programs, many of the therapeutic agents, such as vaccines, are delivered in a dry or lyophilized form. These therapeutic agents must be reconstituted by mixing with sterile water to be placed in condition for injection. Sterile water is often provided in stoppered vials. A syringe with a detachable needle may be used to pierce the vial stopper and draw sterile water into the syringe barrel. The needle is then withdrawn from the stopper and forced through the stopper of another vial containing the lyophilized medication or inserted into the open end of an ampoule containing the lyophilized medication. After these steps, the needle is desirably replaced with a new needle for injection into the patient or a new syringe assembly is provided. This avoids any complications from possible contamination or damage to the needle during the reconstitution process. The removable needle, if not properly disposed of, may be re-used to the detriment of the subsequent user. Also, if the needle is permanently attached to the syringe used in reconstituting the drug, the disposed needle and syringe assembly may also be improperly re-used.

There is a need for a reconstitution syringe assembly capable of reconstituting medication contained in stopper vials or ampoules and having features to prevent unintended re-use for injection.

### SUMMARY OF THE INVENTION

A reconstitution syringe assembly includes an elongate cannula made of thermoplastic material. The cannula has an outside surface, a proximal end, a distal end and a lumen therethrough defining a longitudinal axis. The outside surface at the proximal end of the cannula has at least one discontinuity. The distal end of the cannula includes a stopper piercing tip and has an outside diameter of at least 2 mm. A barrel made of thermoplastic material includes a side wall having an inside surface defining a chamber for retaining fluid, an open proximal end and a distal end including a distal wall with a tip extending distally therefrom having a passageway therethrough in fluid communication with the chamber. The tip of the barrel is formed in intimate contact around the proximal end of the cannula engaging the at least one discontinuity so that the lumen is in fluid communication with the chamber of the barrel. The cannula projects distally from the distal wall of the barrel for a distance of at least 23 mm. The thermoplastic material of the cannula has a higher flexural modulus than the thermoplastic material of the barrel. A plunger including an elongate body portion having a proximal end, a distal end and a stopper at the distal end is provided. The stopper is slidably positioned in fluid-tight engagement with the inside surface of the barrel for drawing fluid into and driving fluid out of the chamber by movement of the stopper relative to the barrel. The elongate body portion of the plunger extends outwardly from the open proximal end of the barrel.

The stopper piercing tip on the cannula preferably includes a closed distal end and at least one side aperture in fluid communication with the lumen. The distal end of the cannula may also have an open end that includes a planar surface at an obtuse angle with respect to the longitudinal axis of the cannula.

It is preferred that the at least one discontinuity includes one or more annular recesses and/or one or more annular projections.

The preferred material for the cannula is polycarbonate and the preferred material of the barrel is polypropylene. The present invention also includes a method of making a barrel and permanently attached cannula for a reconstitution syringe assembly comprising the steps of:
molding an elongate thermoplastic cannula having an outside surface, a proximal end, a distal end and a lumen therethrough defining a longitudinal axis wherein the outside surface of the proximal end includes at least one discontinuity and the distal end includes a stopper piercing tip, said distal end of said cannula having an outside diameter of at least 2 mm (0.08 inch);
molding a thermoplastic barrel over the proximal end of the cannula wherein the barrel includes a side wall having an inside surface defining a chamber for retaining fluid, an open proximal end and a distal end including a distal wall with a tip extending distally therefrom having a passageway in fluid communication with the chamber. The tip is molded in intimate contact around the proximal end of the cannula engaging the at least one discontinuity so that the lumen is in fluid communication with the chamber. The cannula projects distally from the distal wall of the barrel for a distance of at least 23 mm. The thermoplastic material of the cannula has a higher flexural modulus than the thermoplastic material of the barrel.

The preferred thermoplastic material for the cannula is polycarbonate and the preferred thermoplastic material for the barrel is polypropylene.

The tip of the cannula preferably includes a closed distal end and at least one side aperture in fluid communication with the lumen.

The preferred cannula includes the at least one discontinuity being one or more annular recesses and/or one or more annular projections.

### BRIEF DESCRIPTION OF THE DRAWINGS

FIG. 1 is a side elevation view of the syringe assembly of the present invention.

FIG. 2 is a cross-sectional view of the syringe assembly of FIG. 1 taken along line 2-2.

FIG. 3 is an enlarged cross-sectional view of the syringe assembly of FIG. 2 illustrating the distal of the barrel and the proximal end of the permanently attached cannula.

FIG. 4 is a partially cross-sectioned side elevation view illustrating the syringe assembly being used to remove sterile liquid from a stoppered vial.

FIG. 5 is a partially cross-sectioned side elevation view showing the syringe assembly being used to withdraw sterile liquid from a glass ampoule.

FIG. 6 is a side-elevation view of the distal end of a syringe assembly showing an alternative cannula tip.

FIG. 7 is a cross-sectional view of the barrel and cannula of FIG. 6 taken along line 7-7.

FIG. 8 is an enlarged cross-sectional view illustrating the formed barrel and permanently attached cannula in an injection mold.

### DETAILED DESCRIPTION

While this invention is satisfied by embodiments in many different forms, there is shown in the drawings and will herein be described in detail preferred embodiments of the invention with the understanding that the present disclosure is to be considered as exemplary of the principles of the invention and not intended to limit the invention to the embodiments illustrated. The scope of the invention will be measured by the appended claims and their equivalents.

For the purposes of the description of the present invention, the term “distal end” is intended to refer to the end furthest from the person holding the syringe, whereas the term “proximal end” is intended to refer to the end closest to the holder of the syringe.

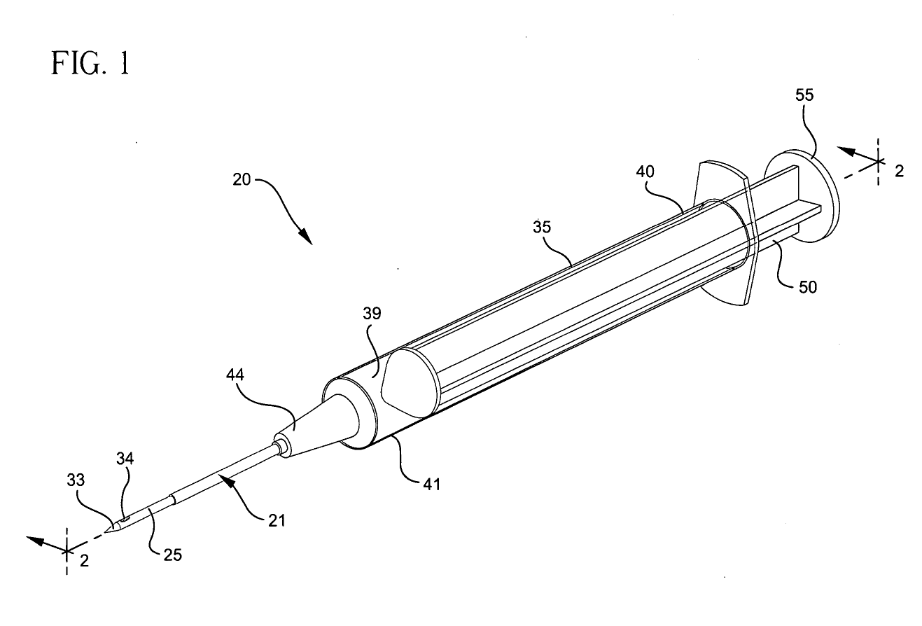

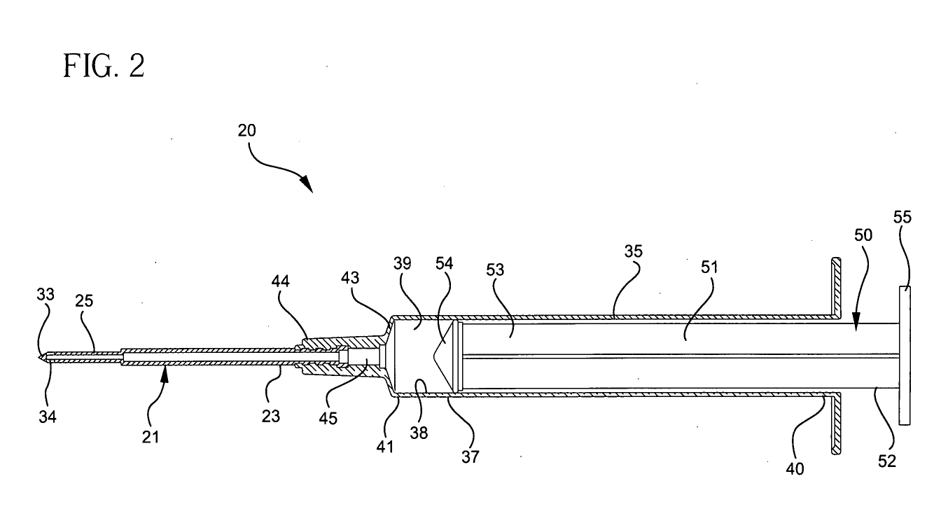

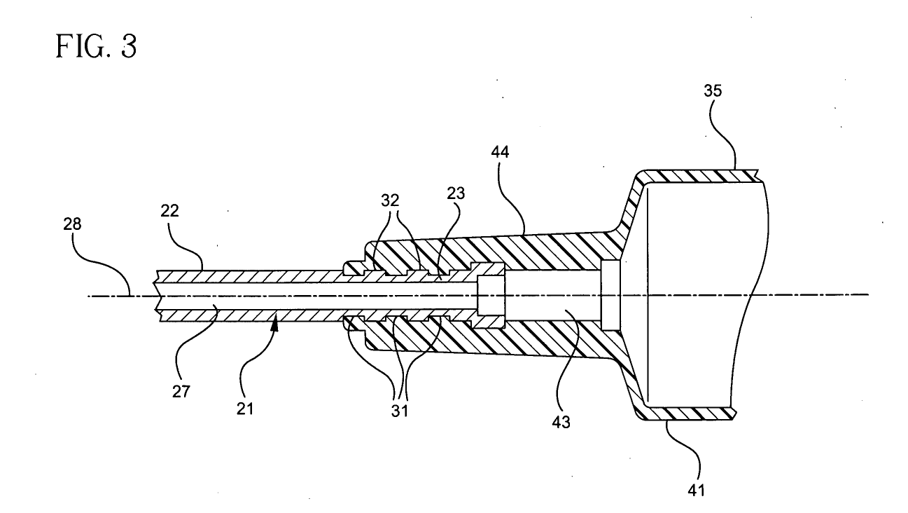

Referring to FIGS. 1-7, a reconstitution syringe assembly 20 includes an elongate cannula 21 preferably made of thermoplastic material. The cannula includes an outside surface 22, a proximal end 23, a distal end 25 and a lumen 27 therethrough defining a longitudinal axis 28. Outside surface 22 at the proximal end of the cannula includes at least one discontinuity. In this embodiment, the at least one discontinuity includes annular grooves 31 and annular projections 32.

The distal end of the cannula includes piercing tip 33. The piercing tip is much less sharp than the tip of a metal hypodermic needle, but still sharp enough to pierce the elastomeric stopper of a medication vial. The cannula at the distal end is much larger than a hypodermic needle intended for injection. In this embodiment, the distal end of the cannula has an outside diameter of at least 2 mm (0.08 inch). The combination of the large diameter distal end of the cannula and the relatively blunt piercing tip results in a cannula that is unsuitable for injection and much less likely to cause accidental skin piercing which could result in injury or transfer of disease. The term “piercing tip” as used herein is intended to encompass the tips of larger than injection needle diameter being configured to pierce elastomeric septums of injection vials and not human skin under normal use. The cannula includes the proximal portion having a diameter larger than the diameter of the distal portion. The diameter of the proximal end is equal or greater than about 2.5 mm (0.1 inch). The increased proximal portion diameter substantially strengthens the cannula when bending forces are applied without, as will be explained hereinafter, interfering with the ability to function properly.

In this embodiment, the distal end of the cannula at the piercing tip is closed and includes at least one side aperture 34 in fluid communication with lumen 27.

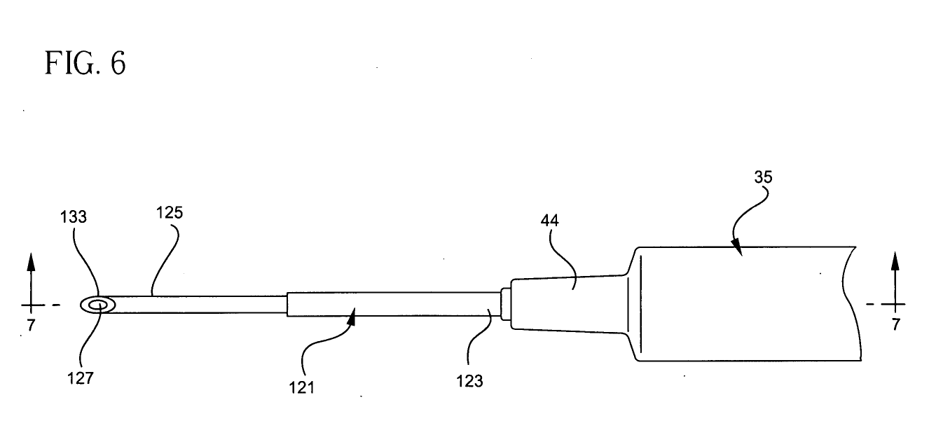

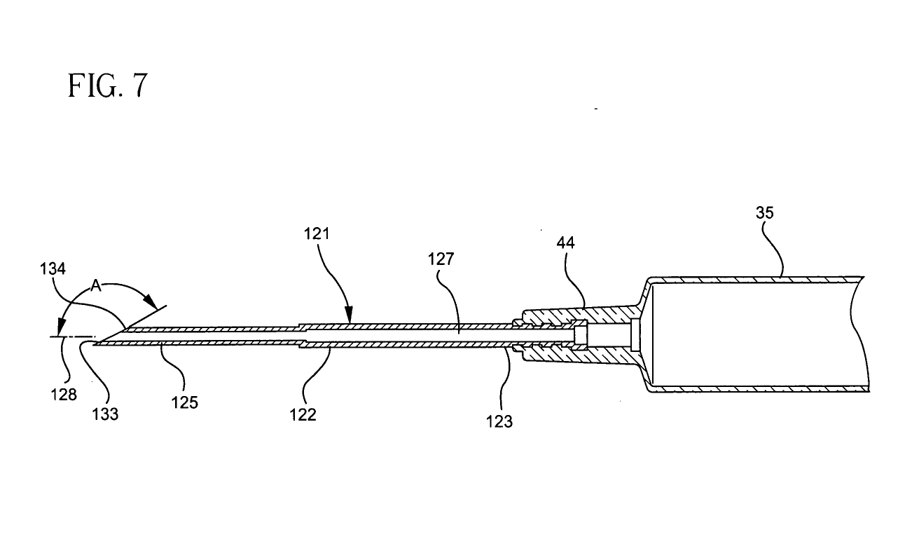

Piercing tips within the purview of the present invention can include other configurations. FIGS. 6 and 7 illustrate an alternative embodiment wherein cannula 121 includes an outside surface 122, a proximal end 123, a distal end 125 and a lumen 127 therethrough defining longitudinal axis 128. A piercing tip 133 at the distal end of the cannula has an outside diameter of at least 2 mm (0.08 inch). The piercing tip has a generally planar surface 134 positioned at an obtuse angle A with respect to longitudinal axis 128.

The syringe assembly also includes a barrel 35 made of thermoplastic material. The barrel includes a side wall 37 having an inside surface 38 defining a chamber 39 for retaining fluid, an open proximal end 40, and a distal end 41 having a distal wall 43 with a tip 44 extending therefrom having a passageway 45 therethrough in fluid communication with the chamber. As will be explained in more detail hereinafter, tip 44 is formed in intimate contact around proximal end 23 of the cannula engaging the annular grooves and projections so that lumen 27 is in fluid communication with chamber 39. Cannula 21 preferably projects distally from the distal wall of the barrel for a distance of at least 23 mm (0.9 inch). In this preferred embodiment cannula 21 extends at least 23 mm (0.9 inch) beyond the distal end of tip 44. The thermoplastic material of the cannula has a higher flexural modulus than the thermoplastic material of the barrel. The flexural modulus of the cannula material is at lest 50% higher than the flexural modulus of the barrel material. In this embodiment, the barrel is preferably formed of polypropylene and the cannula is preferably made of polycarbonate.

A plunger 50 includes an elongate body portion 51 having a proximal end 52, a distal end 53 and a stopper 54 at the distal end. The stopper is slidably positioned in fluid-tight engagement with the inside surface of the barrel for drawing fluid into and driving fluid out of the chamber by moving the stopper relative to the barrel. The elongate body portion of the plunger extends outwardly from the open proximal end of the barrel. A plunger flange 55 is provided on the proximal end of the plunger to facilitate moving the plunger with respect to the barrel. The stopper may be a separate element connected to the body portion of the plunger rod. The separate stopper may be made of thermoplastic materials, thermoplastic elastomers, natural rubber, synthetic rubber and combinations thereof. The stopper in this preferred embodiment is integrally formed with the elongate body portion and it is made of thermoplastic material such as polyethylene.

In the prior art, short hypodermic needle assemblies and short cannula-like spikes are used to withdraw liquid from a stoppered vial. Because these elements are removable, the syringe assembly used in the procedure can be subsequently improperly used with a needle for injecting substances into a person. In the case of a hypodermic needle being used to access the vial, the needle can be improperly used if not properly disposed. The syringe assembly of the present invention eliminates these problems by providing an integral cannula and syringe barrel wherein the cannula has a large piercing tip not suitable for injecting substances into people. Further, it is easier to dispose of since it does not have any metal components. However, an integrally formed syringe and cannula having a short cannula would not be suitable for drawing liquid from a glass ampoule since the ampoule cannot be inverted without spilling the liquid and the cannula must be long enough to reach to the bottom of the ampoule. For these applications, a long hypodermic needle is used. This combination results in the same problems as having a potentially re-usable needle assembly and syringe barrel reusable for human injection as previously described. Further, a long plastic cannula made of commonly used plastics for these applications, such as polypropylene, may bend or become damaged if it were used in an attempt to pierce a stopper vial. This is due to the long length of the cannula which renders it generally undesirable for piercing vials. It is an important aspect of the present invention that all of the above-mentioned problems are overcome by providing a syringe assembly having permanently attached cannula with a relatively large piercing tip which is not suitable for human injection. Further, the cannula is long enough to access vials yet strong enough to pierce vial stoppers to effectively withdraw liquid from a vial. Also, disposal is simplified because there are no metal components in the syringe assembly. The issue of strength is addressed by forming the cannula of a substantially more rigid material than the barrel. Also when the stopper is a rigid element, as in the preferred embodiment, the barrel must be flexible enough to provide a fluid-tight seal around the periphery of the stopper. The more rigid material used in the cannula could not be used in the barrel because the barrel would not have the necessary flexibility to provide an efficacious seal around the stopper and still allow the plunger rod to move with respect to the barrel when reasonable forces are applied. The syringe assembly of the present invention overcomes the deficiencies of the prior art by providing a reconstitution syringe having a rigid plastic cannula with a tip not suitable for human injection which can adequately access stoppered vials and glass ampoules to reconstitute medication and subsequently to be easily destroyed and not be a danger for drug mis-use.

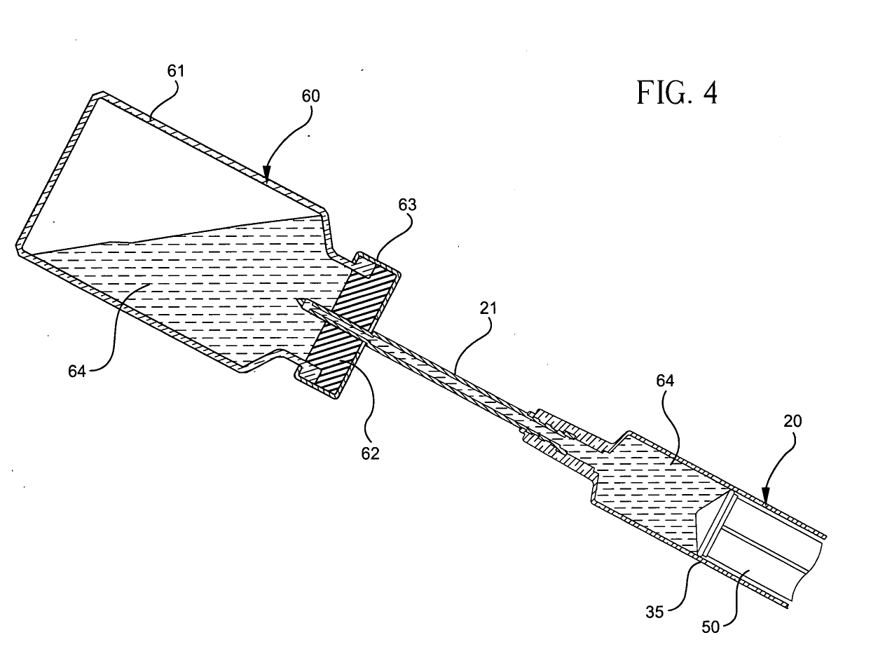

In use, as illustrated in FIG. 4, the syringe assembly of the present invention can be used to reconstitute medications wherein the liquid component is contained in a stoppered vial. As illustrated, stoppered vial 60 includes a vial 61, a pierceable stopper 62, a sheet metal closure 63 for holding the stopper in place on the vial and a quantity of sterile liquid 64. The liquid is drawn into the syringe assembly using known clinically acceptable methods which include piercing the stopper of the vial with the piercing tip of the cannula, injecting a quantity of air into the vial substantially equal to the liquid dose to be withdrawn and moving the plunger in a proximal direction with respect to the barrel to draw liquid into the chamber of the syringe barrel while the vial is inverted so that the short length of cannula can access all of the water in the vial. The syringe assembly 20 with integral cannula 21 is then withdrawn from the stoppered vial and used to transfer the liquid into the dried or lyophilized medication, such as the vaccine, for subsequent injection into the patient. The vaccine may also be contained in a stoppered vial. If so, the integral cannula of the syringe assembly can again be used to pierce the stopper and to force the water into the medication-containing vial for subsequent injection into a patient.

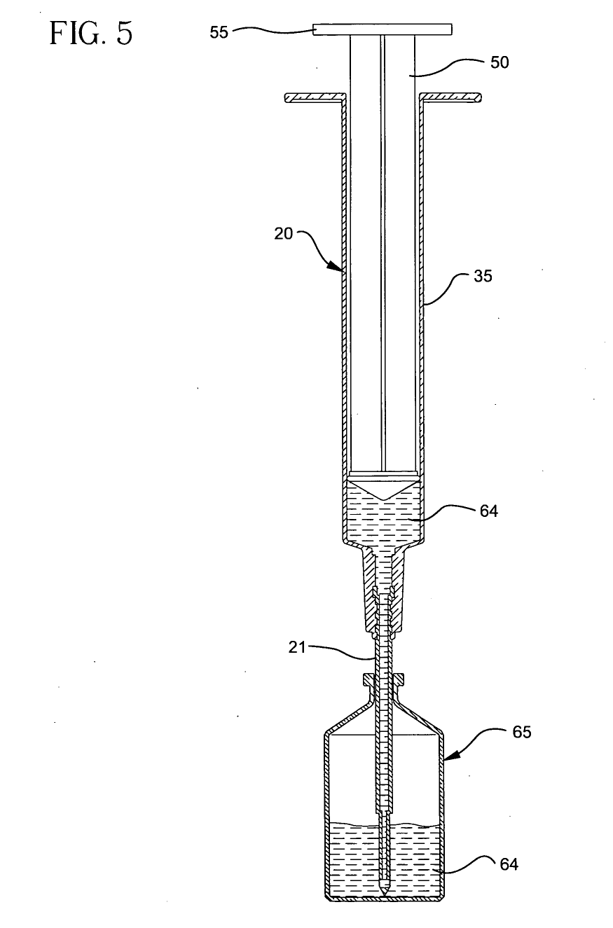

FIG. 5 illustrates syringe assembly 20 being used to withdraw sterile liquid from a glass ampoule 65 containing sterile liquid 64. For this application, the cannula must be small enough to enter the severed neck of the ampoule and long enough to access sterile liquid 64 at the bottom of the ampoule. It is anticipated that a cannula with an effective length of 23 mm (0.9 inch) to 38 mm (1.5 inch) will be able to work with the majority of ampoules believed to be available. It is preferred that the effective length be measured from the distal end of the barrel tip to the distal end of the cannula since it is anticipated that the barrel tip of adequate strength will be much larger than the cannula and not suitable to enter some ampoules. However, if the barrel tip is small enough to fit into the vial the effective length can be measured from the distal wall of the barrel.

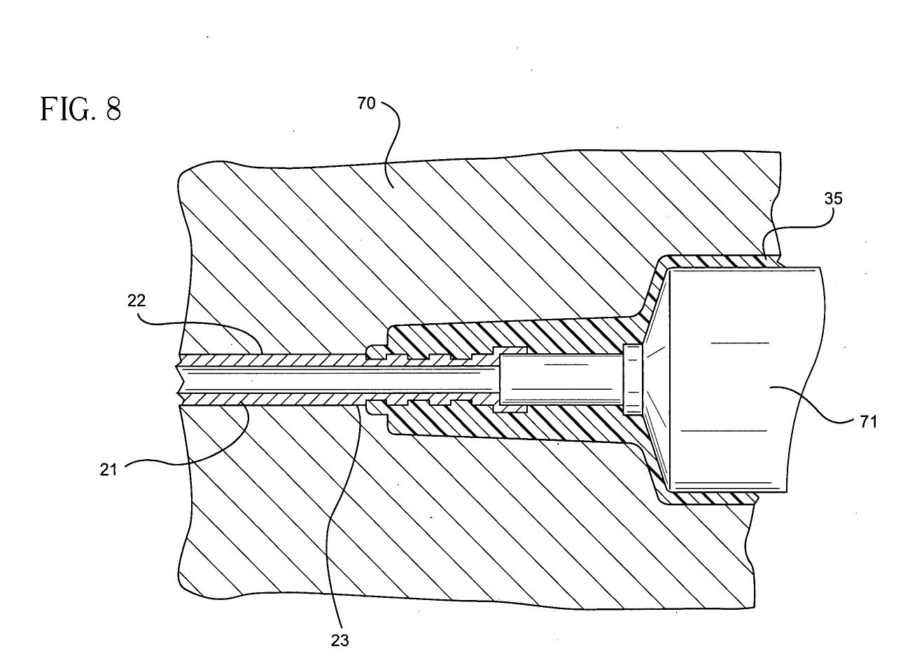

Referring to FIG. 8, the preferred method of making barrel 35 and permanently attached cannula 21 is using an insert molding process. First, the cannula is molded of a relatively rigid material having a higher flexural modulus than the material of the barrel. The next step involves molding the barrel with the molded cannula already in the mold so that barrel tip 44 is formed in intimate contact around proximal end 23 of the cannula and the barrel material engages the discontinuities on outside surface 22 of the cannula. This second step is illustrated in FIG. 8 wherein steel mold 70 and core pin 71 are shown in and around the molded cannula 21 and barrel 35. Upon completion of the molding process the mold is opened and the completed syringe barrel with permanently attached cannula is ejected from the mold.

The flexural modulus of the cannula material, measured in units MPa is at least 50% greater than the flexural modulus of the barrel material. Preferred materials for the barrel and the cannula are polypropylene and polycarbonate respectively. Polycarbonate, having a flexural modulus of about 2275 MPa gives the cannula the substantial strength it needs to function properly and still be long enough to access the full depth of an ampoule and the polypropylene, having a flexural modulus of about 1100 MPa, provides a relatively flexible, less rigid, barrel that will provide an adequate seal for a thermoplastic stopper made of a material such as polyethylene. Also, the shrinkage rate of polypropylene in the molding process is greater than the shrinkage rate of polycarbonate so that as the molded polypropylene which surrounds the proximal end of the polycarbonate cannula solidifies the barrel tip will shrink tightly around the polycarbonate needle to hold it even with more force than if the materials had similar shrinkage rates. Accordingly, even if the polycarbonate cannula is still in the process of solidifying injection molding of the barrel over the cannula is possible.

What is claimed is:

1. A reconstitution syringe assembly comprising:
an elongate cannula made of thermoplastic material, said cannula having an outside surface, a proximal end, a distal end and a lumen therethrough defining a longitudinal axis, said outside surface at said proximal end including at least one discontinuity, said distal end having a stopper piercing tip, said distal end of said cannula having an outside diameter of at least 2 mm (0.08 inch);
a barrel made of thermoplastic material, said barrel including a side wall having an inside surface defining a chamber for retaining fluid, an open proximal end, and a distal end including a distal wall with a tip extending distally therefrom having a passageway therethrough in fluid communication with said chamber, said tip being formed in intimate contact around said proximal end of said cannula engaging said at least one discontinuity so that said lumen is in fluid communication with said chamber, said cannula projecting distally from said distal wall for a distance of at least 23 mm (0.9 inch), said thermoplastic material of said cannula having a higher flexural modulus than said thermoplastic material of said barrel; and
a plunger including an elongate body portion having a proximal end, a distal end and a stopper at said distal end slidably positioned in fluid-tight engagement with said inside surface of said barrel for drawing fluid into and driving fluid out of said chamber by moving said stopper relative to said barrel, said elongate body portion extending outwardly from said open proximal end of said barrel.

2. The syringe of claim 1 wherein said tip of said cannula includes a closed distal end and at least one side aperture in fluid communication with said lumen.

3. The syringe of claim 1 wherein said tip of said cannula includes a planar surface and at an obtuse angle with respect to said longitudinal axis.

4. The syringe of claim 1 wherein said at least one discontinuity includes an annular recess.

5. The syringe of claim 1 wherein said at least one discontinuity includes an annular projection.

6. The syringe assembly of claim 1 wherein said thermoplastic material of said cannula has a flexural modulus of at least 50% greater than the flexural modulus of said thermoplastic material of said barrel.

7. The syringe assembly of claim 1 wherein said cannula is made of polycarbonate.

8. The syringe assembly of claim 1 wherein said barrel is made of polypropylene.

9. The syringe assembly of claim 1 wherein said proximal end of said cannula has an outside diameter of at least 2.5 mm (0.1 inch).

10. The syringe assembly of claim 1 wherein said plunger is integrally formed of thermoplastic material.

11. A reconstitution syringe assembly comprising:
an elongate cannula having an outside surface, a proximal end, a distal end and a lumen therethrough defining a longitudinal axis, said cannula being formed of thermoplastic material, said outside surface at said proximal end including at least one discontinuity, said distal end having a stopper piercing tip including a closed distal end and at least one side aperture in fluid communication with said lumen, said distal end of said barrel having an outside diameter of at least 2 mm (0.08 inch);
a barrel having an inside surface defining a chamber for retaining fluid, an open proximal end, a distal end including a distal wall with a tip extending distally therefrom having a passageway therethrough in fluid communication with said chamber, said barrel being made of thermoplastic material, said tip being formed in intimate contact around said proximal end of said cannula engaging said at least one discontinuity so that said lumen is in fluid communication with said chamber, said cannula projecting distally from said distal wall for a distance of at least 23 mm (0.9 inch), said thermoplastic material of said cannula having a flexural modulus of at least 50% greater than the flexural modulus of said thermoplastic material of said barrel; and
a thermoplastic plunger including an elongate body portion having a proximal end, a distal end and a stopper at said distal end slidably positioned in fluid-tight engagement with said inside surface of said barrel for drawing fluid into and driving fluid out of said chamber by moving said stopper relative to said barrel, said elongate body portion extending outwardly from said open end of said barrel.

12. A method of making a barrel and permanently attached cannula for a reconstitution syringe assembly comprising the steps of:
molding an elongate thermoplastic cannula having an outside surface, a proximal end, a distal end and a lumen therethrough defining a longitudinal axis, said outside surface of said proximal end including at least one discontinuity, said distal end having a stopper piercing tip, said distal end of said cannula having an outside diameter of at least 2 mm (0.08 inch),
molding a thermoplastic barrel over said proximal end of said cannula, said barrel including a side wall having an inside surface defining a chamber for retaining fluid, an open proximal end, and a distal end including a distal wall with a tip extending distally therefrom having a passageway in fluid communication with said chamber, said tip being molded in intimate contact around said proximal end of said cannula engaging said at least one discontinuity so that the lumen is in fluid communication with said chamber, said cannula projecting distally from said distal wall for a distance of at least 23 mm (0.9 inch), said thermoplastic material of said cannula having a flexural modulus at least 50% greater than the flexural modulus of said thermoplastic material of said barrel.

13. The method of claim 12 wherein said cannula is molded of polycarbonate material.

14. The method of claim 12 wherein the barrel is made of polypropylene.

15. The method of claim 12 wherein said tip of said cannula includes a closed distal end and at least one side aperture in fluid communication with said lumen.

16. The method of claim 12 wherein said at least one discontinuity includes an annular recess.

17. The method of claim 12 wherein said at least one discontinuity includes an annular projection.
